(19) United States
(12) Patent Application Publication
(10) Pub. No.: US 2011/0092487 A1
(43) Pub. Date: Apr. 21, 2011

(54) NOVEL 3,8-DIAZA-BICYCLO[3.2.1]OCTANE-AND 3,9-DIAZA-BICYCLO[3.3.1]-NONANE-3-CARBOXYLIC ACID ESTER DERIVATIVES AND THEIR USE AS MONOAMINE NEUROTRANSMITTER RE-UPTAKE INHIBITORS

(75) Inventors: Dan Peters, Malmö (SE); John Paul Redrobe, Rodovre (DK); Elsebet Østergaard Nielsen, København K (DK)

(73) Assignee: NeuroSearch A/S, Ballerup (DK)

(21) Appl. No.: 12/920,965

(22) PCT Filed: Feb. 27, 2009
(86) PCT No.: PCT/EP2009/052329

§ 371 Date: Dec. 6, 2010

Related U.S. Application Data
(60) Provisional application No. 61/034,266, filed on Mar. 6, 2008.

(30) Foreign Application Priority Data
Mar. 5, 2008 (DK) PA 2008 00324

(51) Int. Cl. A61K 31/55; C07D 471/08; A61K 31/4995; A61P 25/00; A61P 25/24; A61P 25/22; A61P 3/04; A61P 25/16; A61P 25/28; A61P 25/30

(52) U.S. Cl. 514/215; 540/477; 540/580; 514/249

(57) ABSTRACT

This invention relates to novel 3,8-diaza-bicyclo[3.2.1]octane-and 3,9-diaza-bicyclo[3.3.1]nonane-3-carboxylic acid ester derivatives useful as monoamine neurotransmitter re-uptake inhibitors. In other aspects the invention relates to the use of these compounds in a method for therapy and to pharmaceutical compositions comprising the compounds of the invention. Formula (I).

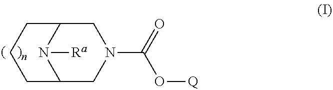

### TECHNICAL FIELD

This invention relates to novel 3,8-diaza-bicyclo[3.2.1]octane- and 3,9-diaza-bicyclo[3.3.1]nonane-3-carboxylic acid ester derivatives useful as monoamine neurotransmitter re-uptake inhibitors.

In other aspects the invention relates to the use of these compounds in a method for therapy and to pharmaceutical compositions comprising the compounds of the invention.

### BACKGROUND ART

Serotonin Selective Reuptake Inhibitors (SSRIs) currently provide efficacy in the treatment of several CNS disorders, including depression and panic disorder. SSRIs are generally perceived by psychiatrists and primary care physicians as effective, well-tolerated and easily administered. However, they are associated with a number of undesirable features.

Thus, there is still a strong need for compounds with an optimised pharmacological profile as regards the activity on reuptake of the monoamine neurotransmitters serotonin, dopamine and noradrenaline, such as the ratio of the serotonin reuptake versus the noradrenaline and dopamine reuptake activity.

Fontanella Let al [Farmaco, Edizione Scientifica (1975), 30(9), 742-53] discloses the compound 8-methyl-3,8-diaza-bicyclo[3.2.1]octane-3-carboxylic acid phenyl ester.

### SUMMARY OF THE INVENTION

It is an object of the invention to provide novel compounds which show activity as monoamine neurotransmitter re-uptake inhibitors.

In its first aspect, the invention provides a compound of Formula I:

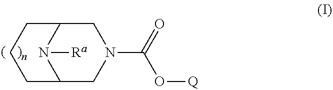

any of its stereoisomers or any mixture of its stereoisomers, or an N-oxide thereof, or a pharmaceutically acceptable salt thereof, wherein n, R^{a} and Q are as defined below.

In its second aspect, the invention provides a pharmaceutical composition, comprising a therapeutically effective amount of a compound of the invention, any of its stereoisomers or any mixture of its stereoisomers, or an N-oxide thereof, or a pharmaceutically acceptable salt thereof, together with at least one pharmaceutically acceptable carrier, excipient or diluent.

In a further aspect, the invention provides the use of a compound of the invention, any of its stereoisomers or any mixture of its stereoisomers, or an N-oxide thereof, or a pharmaceutically acceptable salt thereof, for the manufacture of a pharmaceutical composition for the treatment, prevention or alleviation of a disease or a disorder or a condition of a mammal, including a human, which disease, disorder or condition is responsive to inhibition of monoamine neurotransmitter re-uptake in the central nervous system.

In a still further aspect, the invention relates to a method for treatment, prevention or alleviation of a disease or a disorder or a condition of a living animal body, including a human, which disorder, disease or condition is responsive to responsive to inhibition of monoamine neurotransmitter re-uptake in the central nervous system, which method comprises the step of administering to such a living animal body in need thereof a therapeutically effective amount of a compound of the invention, any of its stereoisomers or any mixture of its stereoisomers, or an N-oxide thereof, or a pharmaceutically acceptable salt thereof.

Other objects of the invention will be apparent to the person skilled in the art from the following detailed description and examples.

### DETAILED DISCLOSURE OF THE INVENTION

In its first aspect the present invention provides compounds of Formula I:

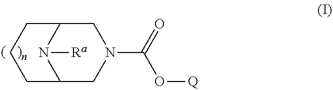

any of its stereoisomers or any mixture of its stereoisomers, or an N-oxide thereof, or a pharmaceutically acceptable salt thereof, wherein
n is 0 or 1;
R^{a} represents hydrogen or C₁₋₆-alkyl;
Q represents an aryl group or a chromen-2-one group; which aryl group or chromen-2-one group is optionally substituted with one or more substituents independently selected from the group consisting of halo, trifluoromethyl, trifluoromethoxy, cyano, hydroxy, amino, nitro, C₁₋₆-alkoxy, C₃₋₇-cycloalkoxy, C₁₋₆-alkyl, C₃₋₇-cycloalkyl, C₃₋₇-cycloalkyl-C₁₋₆-alkyl, C₂₋₆-alkenyl and C₂₋₆-alkynyl;
and with the proviso that the compound is not 8-methyl-3,8-diaza-bicyclo[3.2.1]octane-3-carboxylic acid phenyl ester.

In one embodiment of the compound of Formula I, n is 0. In another embodiment of the compound of Formula I, n is 1.

In another embodiment of the compound of Formula I, R^{a} represents hydrogen. In another embodiment of the compound of Formula I, R^{a} represents C₁₋₆-alkyl, such as methyl.

In another embodiment of the compound of Formula I, Q represents an optionally substituted phenyl group. In another embodiment, Q represents a substituted phenyl group. In another embodiment, Q represents halophenyl, such as 4-halo-phenyl. In another embodiment, Q represents bromophenyl, such as 4-bromophenyl. In another embodiment, Q represents dihalophenyl, such as 3,4-dihalophenyl. In another embodiment, Q represents dichlorophenyl, such as 3,4-dichlorophenyl.

In another embodiment of the compound of Formula I, Q represents an optionally substituted naphthyl group, such as an optionally substituted naphthalene-2-yl group. In another embodiment, Q represents a substituted naphthyl group, such as a substituted naphthalene-2-yl group. In another embodiment, Q represents C₁₋₆-alkoxynaphthyl, such as methoxynaphthyl. In another embodiment, Q represents 6-alkoxynaphthyl, such as 6-alkoxynaphthalen-2-yl, such as 6-methoxynaphthalen-2-yl.

In another embodiment of the compound of Formula I, Q represents an optionally substituted chromen-2-one group. In another embodiment of the compound of formula I, Q represents chromen-2-one-yl, such as chromen-2-one-6-yl or chromen-2-one-7-yl. In another embodiment, Q represents substituted chromen-2-one-yl, such as halosubstituted chromen-2-one-yl, such as bromosubstituted chromen-2-one-yl. In another embodiment, Q represents chromen-2-one-yl; which chromen-2-one-yl group is substituted in the 3-position with halo, such as bromo. In another embodiment, Q represents 3-bromo-chromen-2-one-7-yl.

In another embodiment, the compound of the invention is

9-Methyl-3,9-diaza-bicyclo[3.3.1]nonane-3-carboxylic acid 3,4-dichloro-phenyl;
9-Methyl-3,9-diaza-bicyclo[3.3.1]nonane-3-carboxylic acid 4-bromo-phenyl ester;
9-Methyl-3,9-diaza-bicyclo[3.3.1]nonane-3-carboxylic acid 6-methoxynaphthalen-2-yl ester;
9-Methyl-3,9-diaza-bicyclo[3.3.1]nonane-3-carboxylic acid 2-oxo-2H-chromen-7-yl ester;
9-Methyl-3,9-diaza-bicyclo[3.3.1]nonane-3-carboxylic acid 2-oxo-2H-chromen-6-yl ester;
8-Methyl-3,8-diaza-bicyclo[3.2.1]octane-3-carboxylic acid 4-bromo-phenyl ester;
8-Methyl-3,8-diaza-bicyclo[3.2.1]octane-3-carboxylic acid 2-oxo-2H-chromen-7-yl ester;
8-Methyl-3,8-diaza-bicyclo[3.2.1]octane-3-carboxylic acid 2-oxo-2H-chromen-6-yl ester;
3,9-Diaza-bicyclo[3.3.1]nonane-3-carboxylic acid 3,4-dichloro-phenyl ester;
3,9-Diaza-bicyclo[3.3.1]nonane-3-carboxylic acid 2-oxo-2H-chromen-7-yl ester;
3,8-Diaza-bicyclo[3.2.1]octane-3-carboxylic acid 2-oxo-2H-chromen-7-yl ester;
9-Methyl-3,9-diaza-bicyclo[3.3.1]nonane-3-carboxylic acid 3-bromo-2-oxo-2H-chromen-7-yl ester;
or a pharmaceutically acceptable salt thereof.

Any combination of two or more of the embodiments as described above is considered within the scope of the present invention.

### Definition of Substituents

As used throughout the present specification and appended claims, the following terms have the indicated meaning:

The term “C₁₋₆-alkyl” as used herein means a saturated, branched or straight hydrocarbon group having from 1-6 carbon atoms, e.g. C₁₋₃-alkyl, C₁₋₄-alkyl, C₁₋₆-alkyl, C₂₋₆-alkyl, C₃₋₆-alkyl, and the like. Representative examples are methyl, ethyl, propyl (e.g. prop-1-yl, prop-2-yl (or iso-propyl)), butyl (e.g. 2-methylprop-2-yl (or tert-butyl), but-1-yl, but-2-yl), pentyl (e.g. pent-1-yl, pent-2-yl, pent-3-yl), 2-methylbut-1-yl, 3-methylbut-1-yl, hexyl (e.g. hex-1-yl), and the like.

The term “C₂₋₆-alkenyl” as used herein represents a branched or straight hydrocarbon group having from 2 to 6 carbon atoms and at least one double bond, e.g. C₂₋₆-alkenyl, C₃₋₆-alkenyl, and the like. Representative examples are ethenyl (or vinyl), propenyl (e.g. prop-1-enyl, prop-2-enyl), butadienyl (e.g. buta-30 1,3-dienyl), butenyl (e.g. but-1-en-1-yl, but-2-en-1-yl), pentenyl (e.g. pent-1-en-1-yl, pent-2-en-2-yl), hexenyl (e.g. hex-1-en-2-yl, hex-2-en-1-yl), 1-ethylprop-2-enyl, 1,1-(dimethyl)prop-2-enyl, 1-ethylbut-3-enyl, 1,1-(dimethyl)but-2-enyl, and the like.

The term “C₂₋₆-alkynyl” as used herein represents a branched or straight hydrocarbon group having from 2 to 6 carbon atoms and at least one triple bond. Representative examples are ethynyl, propynyl (e.g. prop-1-ynyl, prop-2-ynyl), butynyl (e.g. but-1-ynyl, but-2-ynyl), pentynyl (e.g. pent-1-ynyl, pent-2-ynyl), hexynyl (e.g. hex-1-ynyl, hex-2-ynyl), 1-ethylprop-2-ynyl, 1,1-(dimethyl)prop-2-ynyl, 1-ethylbut-3-ynyl, 1,1-(dimethyl)but-2-ynyl, and the like.

The term “halo” or “halogen” shall mean fluorine, chlorine, bromine or iodine.

The term “hydroxy” shall mean the radical —OH.

The term “cyano” shall mean the radical —CN.

The term “nitro” shall mean the radical —NO₂.

The term “amino” shall mean the radical —NH₂ or NH—C₁₋₆-alkyl or N-(C₁₋₆-alkyl)₂, wherein C₁₋₆-alkyl is as defined above.

The term “trihalomethyl” shall mean trifluoromethyl, trichloromethyl, and similar trihalo-substituted methyl groups.

The term “trihalomethoxy” shall mean trifluoromethoxy, trichloromethoxy, and similar trihalo-substituted methoxy groups.

The term “C₁₋₆-alkoxy” as used herein refers to the radical alkyl-O-. Representative examples are methoxy, ethoxy, propoxy (e.g. 1-propoxy, 2-propoxy), butoxy (e.g. 1-butoxy, 2-butoxy, 2-methyl-2-propoxy), pentoxy (1-pentoxy, 2-pentoxy), hexoxy (1-hexoxy, 3-hexoxy), and the like.

The term “C₃₋₇-cycloalkyl” as used herein represents a saturated monocyclic carbocyclic ring having from 3 to 7 carbon atoms, e.g. C₃₋₆-alkyl, C₃₋₄-alkyl, C₃₋₅-alkyl C₃₋₇-alkyl, and the like. Representative examples are cyclopropyl, cyclobutyl, cyclopentyl, cyclohexyl, cycloheptyl, and the like.

The term “C₃₋₇-cycloalkoxy” as used herein represents an C₃₋₇-cycloalkyl group as defined above attached through an oxygen bridge. Representative examples are cyclopropyloxy, cyclobutyloxy, cyclopentyloxy, cyclohexyloxy, cycloheptyloxy, and the like.

The term “aryl” as used herein represents a carbocyclic aromatic ring system such as phenyl, naphthyl (1-naphthyl or 2-naphthyl) or fluorenyl.

Certain of the defined terms may occur in combinations, and it is to be understood that the first mentioned radical is a substituent on the subsequently mentioned radical, where the point of substitution, i.e. the point of attachment to another part of the molecule, is on the last mentioned of the radicals. Such combinations of terms include for example:

“C₁₋₆-alkoxy-C₁₋₆-alkyl”. Representative examples are methoxymethyl, ethoxymethyl, 2-methoxyethyl, 2-ethoxyethyl, 3-methoxyprop-1-yl, and the like.

“C₃₋₇-cycloalkyl-C₁₋₆-alkyl”. Representative examples are cyclopropylmethyl, 2-cyclohexylethyl, 3-cyclopentylprop-1-yl, 1-cyclohexylethyl, adamantylmethyl, and the like.

### Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Examples of pharmaceutically acceptable cationic salts of a chemical compound of the invention include, without limitation, the sodium, the potassium, the calcium, the magnesium, the zinc, the aluminium, the lithium, the choline, the lysinium, and the ammonium salt, and the like, of a chemical compound of the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the chemical compound of the invention include examples of suitable prodrugs of the substances according to the invention include compounds modified at one or more reactive or derivatizable groups of the parent compound. Of particular interest are compounds modified at a carboxyl group, a hydroxyl group, or an amino group. Examples of suitable derivatives are esters or amides.

The chemical compound of the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

### Steric Isomers

It will be appreciated by those skilled in the art that the compounds of the present invention may exist in different stereoisomeric forms—including enantiomers, diastereomers or cis-trans-isomers.

The invention includes all such isomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid—by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L-(tartrates, mandelates, or camphorsulphonate) salts for example.

The chemical compounds of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

### N-Oxides

In the context of this invention an N-oxide designates an oxide derivative of a nitrogen containing compound, e.g. N-containing heterocyclic compounds capable of forming such N-oxides, and compounds holding one or more amino groups. For example, the N-oxide of a compound containing a pyridyl may be the 1-oxypyridin-2, -3 or -4-yl derivative.

N-oxides of the compounds of the invention may be prepared by oxidation of the corresponding nitrogen base using a conventional oxidizing agent such as hydrogen peroxide in the presence of an acid such as acetic acid at an elevated temperature, or by reaction with a peracid such as peracetic acid in a suitable solvent, e.g. dichloromethane, ethyl acetate or methyl acetate, or in chloroform or dichloromethane with 3-chloroperoxybenzoic acid.

### Labelled Compounds

The compounds of the invention may be used in their labelled or unlabelled form. In the context of this invention the labelled compound has one or more atoms replaced by an atom having an atomic mass or mass number different from the atomic mass or mass number usually found in nature. The labelling will allow easy quantitative detection of said compound.

The labelled compounds of the invention may be useful as diagnostic tools, radio tracers, or monitoring agents in various diagnostic methods, and for in vivo receptor imaging.

The labelled isomer of the invention preferably contains at least one radionuclide as a label. Positron emitting radionuclides are all candidates for usage. In the context of this invention the radionuclide is preferably selected from ²H (deuterium), ³H (tritium), ¹¹C, ¹³C, ¹⁴C, ¹³¹I, ¹²⁵I, ¹²³I, and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

### Methods of Preparation

The chemical compounds of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

Compounds of the invention may be tested for their ability to inhibit reuptake of the monoamines dopamine, noradrenaline and serotonin in synaptosomes e.g. such as described in WO 97/30997 (NeuroSearch NS) or WO 97/16451 (NeuroSearch NS). Based on the balanced activity observed in these tests the compound of the invention is considered useful for the treatment, prevention or alleviation of a disease or a disorder or a condition of a mammal, including a human, which disease, disorder or condition is responsive to inhibition of monoamine neurotransmitter re-uptake in the central nervous system.

In a special embodiment, the compounds of the invention are considered useful for the treatment, prevention or alleviation of: mood disorder, depression, atypical depression, depression secondary to pain, major depressive disorder, dysthymic disorder, bipolar disorder, bipolar I disorder, bipolar II disorder, cyclothymic disorder, mood disorder due to a general medical condition, substance-induced mood disorder, pseudodementia, Ganser's syndrome, obsessive compulsive disorder, panic disorder, panic disorder without agoraphobia, panic disorder with agoraphobia, agoraphobia without history of panic disorder, panic attack, memory deficits, memory loss, attention deficit hyperactivity disorder (ADHD), obesity, anxiety, generalized anxiety disorder, eating disorder, Parkinson's disease, parkinsonism, dementia, dementia of ageing, senile dementia, Alzheimer's disease, Down's syndrome, acquired immunodeficiency syndrome dementia complex, memory dysfunction in ageing, specific phobia, social phobia, social anxiety disorder, post-traumatic stress disorder, acute stress disorder, drug addiction, drug abuse, drug abuse liability, cocaine abuse, nicotine abuse, tobacco abuse, alcohol addiction, alcoholism, kleptomania, withdrawal symptoms caused by termination of use of addictive substances, pain, chronic pain, inflammatory pain, neuropathic pain, migraine pain, tension-type headache, chronic tension-type headache, pain associated with depression, fibromyalgia, arthritis, osteoarthritis, rheumatoid arthritis, back pain, cancer pain, irritable bowel pain, irritable bowel syndrome, post-operative pain, post-mastectomy pain syndrome (PMPS), post-stroke pain, drug-induced neuropathy, diabetic neuropathy, sympathetically-maintained pain, trigeminal neuralgia, dental pain, myofacial pain, phantom-limb pain, bulimia, premenstrual syndrome, premenstrual dysphoric disorder, late luteal phase syndrome, post-traumatic syndrome, chronic fatigue syndrome, persistent vegetative state, urinary incontinence, stress incontinence, urge incontinence, nocturnal incontinence, sexual dysfunction, premature ejaculation, erectile difficulty, erectile dysfunction, premature female orgasm, restless leg syndrome, periodic limb movement disorder, eating disorders, anorexia nervosa, sleep disorders, pervasive developmental disorders, autism, Asperger's disorder, Rett's disorder, childhood disintegrative disorder, learning disabilities, motor skills disorders, mutism, trichotillomania, narcolepsy, post-stroke depression, stroke-induced brain damage, stroke-induced neuronal damage, Gilles de la Tourettes disease, tinnitus, tic disorders, body dysmorphic disorders, oppositional defiant disorder or post-stroke disabilities. In another special embodiment, the compounds are considered useful for the treatment, prevention or alleviation of depression. In another special embodiment, the compounds are considered useful for the treatment, prevention or alleviation of attention deficit hyperactivity disorder (ADHD).

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred compounds of the invention show a biological activity in the sub-micromolar and micromolar range, i.e. of from below 1 to about 100 μM.

### Pharmaceutical Compositions

In another aspect the invention provides novel pharmaceutical compositions comprising a therapeutically effective amount of the chemical compound of the invention.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In one embodiment, the invention provides pharmaceutical compositions comprising the chemical compound of the invention, or a pharmaceutically acceptable salt or derivative thereof, together with one or more pharmaceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

In one embodiment, the invention provides tablets or capsules for oral administration.

In another embodiment, the invention provides liquids for intravenous administration and continuous infusion.

Further details on techniques for formulation and administration may be found in the latest edition of Remington's Pharmaceutical Sciences (Maack Publishing Co., Easton, Pa.).

The dose administered must of course be carefully adjusted to the age, weight and condition of the individual being treated, as well as the route of administration, dosage form and regimen, and the result desired, and the exact dosage should of course be determined by the practitioner.

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Ranges are from about 0.1 μg/kg to about 10 mg/kg/day i.v., and from about 1 μg/kg to about 100 mg/kg/day p.o.

### Methods of Therapy

In another aspect the invention provides a method for the treatment, prevention or alleviation of a disease or a disorder or a condition of a living animal body, including a human, which disease, disorder or condition is responsive to inhibition of monoamine neurotransmitter re-uptake in the central nervous system, and which method comprises administering to such a living animal body, including a human, in need thereof an effective amount of a chemical compound of the invention.

It is at present contemplated that suitable dosage ranges are 0.1 to 1000 10 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

### EXAMPLES

The following examples and general procedures refer to intermediate compounds and final products for general formula (I) identified in the specification. The preparation of the compounds of general formula (I) of the present invention is described in detail using the following examples. Occasionally, the reaction may not be applicable as described to each compound included within the disclosed scope of the invention. The compounds for which this occurs will be readily recognized by those skilled in the art. In these cases the reactions can be successfully performed by conventional modifications known to those skilled in the art, which is, by appropriate protection of interfering groups, by changing to other conventional reagents, or by routine modification of reaction conditions. Alternatively, other reactions disclosed herein or otherwise conventional will be applicable to the preparation of the corresponding compounds of the invention. In all preparative methods, all starting materials are known or may easily be prepared from known starting materials.

All reactions involving air sensitive reagents or intermediates are performed under nitrogen and in anhydrous solvents. Magnesium sulphate is used as drying agent in the workup-procedures and solvents are evaporated under reduced pressure.

Diethyl meso-2,6-dibromopimeloate (Intermediate Compound 1)

Pimelic acid (240 g, 1.5 mol) was placed into a two-necked round bottom flask (1000 ml) fitted with a reflux condenser and an argon inlet. The reflux condenser was connected with two consecutive flasks (500 and 1000 ml). The first flask (500 ml) was placed in to dry ice-isopropanol vessel and the second was half filled with water for HCl absorption. Thionyl chloride (368 g, 3.09 mol) was added in three portions (180, 100 and 88 g) and stirred at 40° C. until gas elution ceased. Finally temperature was raised to 100° C., the first flask with liquid SO₂ was disconnected. The flask was fitted with dropping funnel and gas outlet. During 3 hours the flask was continuously irradiated with 300 W UV lamp and bromine (490 g, 3.06 mol) was added drop-wise. The HBr formed was absorbed in two consecutive water filled flasks (2×1000 ml). When HBr elution ceased, the dropping funnel was filled with absolute ethanol (200 ml) and carefully added drop-wise. The chilled solution was washed with water, aqueous sodium acetate and sodium thiosulfate. The separated organic phase was dried over sodium sulfate, filtrated and distilled in multiple portions (about 40 ml each) by a Büchi oven in vacuo (0.5-1.0 mbar) at 150° C. collecting the fraction from the third flask. Yield: 487 g (87%).

Diethyl cis-1-Methylpiperidine-2,6-dicarboxylate (Intermediate Compound 2)

Diethyl meso-2,6-dibromoadipoate (1) (236 g, 0.631 mol) was placed into a two necked round bottom flask (2000 ml) fitted with a reflux condenser and a thermometer, and was dissolved in absolute THF (400 ml) under argon. A solution of methylamine (62 g, 2.0 mol) in absolute THF (400 ml) was added to the solution of compound 1. The flask was placed in cold water, to prevent it from warming. The reaction mixture was stirred for 18 hours under argon, the separated N-methylammonium bromide was removed by filtration and washed thoroughly with THF. The filtrate was concentrated on a rotary evaporator under reduced pressure and the residue (156 g) was distilled in four portions (about 39 g each) by a Büchi oven in vacuo (0.1-0.4 mbar) at 125° C. (average distillation time 1 hour) collecting the fraction from the third flask. Yield of compound 2 127.5 g (83%) as a light-yellowish oil.

3-Benzyl-9-methyl-3,9-diazabicyclo[3.3.1]nonane-2,4-dione (Intermediate Compound 3)

A solution of diethyl cis-1-methylpiperidin-2,6-carboxylate (127.5 g, 0.524 mol) and benzylamine (57.8 g, 0.540 mol) in xylene (150 ml) was refluxed in a round-bottomed flask (250 ml) for 44 hours. The latter was equipped with a vertical air condenser (15 cm) followed by a Liebig condenser, allowing removal of ethanol from the reaction mixture. The xylene was removed under reduced pressure through a Liebig condenser, the oil bath temperature was elevated to 205° C. and the mixture was heated under argon for 20 hours. The obtained product was distilled in four portions (about 45 g each) by a Büchi oven in vacuo (0.1 mbar) at 160° C. (average distillation time 1 hours) collecting the fraction from the third flask. The three combined 3^{rd} fractions (96 g) were dissolved by boiling in 50 ml of ethyl acetate and allowed to crystallize at room temperature for 3 days. The crystalline material was filtered off, washed with a small amount of ethyl acetate and dried in vacuo to afford 39.5 g of the product as a white crystalline solid. The filtrate was concentrated and the residue crystallized from ethyl acetate (30 ml) at 4° C. for 2 days to yield 6.2 g of the same product. Yield of compound 3 was 45.7 g (34%), mp. 117-118° C.

3-Benzyl-9-methyl-3,9-diazabicyclo[3.3.1]nonane (Intermediate Compound 4)

To a solution of compound 3 (45.7 g, 0.177 mol) in 1,4-dioxane (400 ml) placed into a three-necked round bottom flask (1000 ml), LiAlH₄ (9.0 g, 0.237 mol) was added in small portions and the mixture was refluxed under argon for 18 hours. The reaction mixture was cooled to 80° C. and a mixture of water (9 ml) and 1,4-dioxane (40 ml) was dropped carefully into reaction flask (caution: vigorous hydrogen evolution). A fine suspension was cooled to room temperature and treated with KOH solution (20 g in 50 ml of water). The organic phase was decanted and concentrated was concentrated under reduced pressure. The residue was distilled on Büchi oven in vacuo (0.1 mbar) at 130° C. The third collecting flask contained 3,9-diazabicyclo[3.3.1]nonane 4 (29.2 g, 72%) as a viscous colourless oil.

9-Methyl-3,9-diazabicyclo[3.3.1]nonane (Intermediate Compound 5)

To a solution of compound 4 (28.7 g, 0.125 mol) in absolute ethanol (100 30 ml) was added 10% Pd/C catalyst (6.3 g) under argon. The solution was hydrogenated with H₂ at 60 bar and 100° C. for 16 hours. The solution was filtered of on a Büchner funnel, the filtrate was concentrated under reduced pressure on a rotary evaporator and the residue distilled on Büchi oven in vacuo (0.1 mbar) at 100° C. to afford compound 5 (8.5 g, 49%) as a colourless gel.

Diethyl cis-1-methylpyrrolidine-2,5-dicarboxylate (Intermediate Compound)

Diethyl mezo-2,5-dibromoadipate (101.7 g; 0.283 mol) was dissolved by heating under argon in THF (400 ml) and then cooled to 0° C. To the obtained solution a pre-cooled solution of methylamine (27.3 g; 0.88 mol) in THF (150 ml) was added and the mixture was stirred at room temperature for 18 hours. The separated crystalline material was filtered off, the filtrate concentrated and the residue chromatographed on a silica gel column (10 cm long) with hexane-ethyl acetate 4:1 as eluent to afford 58.9 g (91%).

### ¹H NMR (300 MHz, CDCl₃): 1.15 (t, 6H); 1.9-2.0 (m, 4H); 2.38 (s, 3H); 2.99 (m, 2H); 4.07 (q, 4H). ¹³C l NMR (75 MHz, CDCl₃): 13.98; 27.68; 40.82; 60.39; 67.93; 68.06; 172.32.

3-Benzyl-8-methyl-3,8-diazabicyclo[3.2.1]octane-2,4-dione (Intermediate Compound)

To a solution of diethyl cis-1-methylpyrrolidine-2,5-dicarboxylate (74.8 g; 0.383 mol) in xylene (150 ml) benzylamine (41.0 g; 0.383 mol) was added and the mixture heated to reflux for 16 hours. Then xylene was removed at reduced pressure and the residue was heated at 220° C. for 18 hours. The obtained crude product was distilled by portions (30-40 g) on Büchi oven for distillation at 180° C. and 0.1 mbar, and the first fraction collected (after about 1 hours). The combined first fractions were crystallized from a mixture of hexane and ethyl acetate 1:1 to yield 30.6 (34%).

### ¹H NMR (300 MHz, CDCl₃): 1.88 (m, 2H); 2.34 (m, 2H); 2.42 (s, 3H); 3.80 (dd, 2H); 4.88 (s, 2H); 7.2-7.4 (m, 5H). ¹³C NMR (75 MHz, CDCl₃): 26.69; 35.82; 41.26; 65.72; 127.42; 128.36; 128.62; 136.91; 173.26.

3-Benzyl-8-methyl-3,8-diazabicyclo[3.2.1]octane (Intermediate Compound)

To a solution of 3-benzyl-8-methyl-3,8-diazabicyclo[3.2.1]octane-2,4-dione (28.3 g; 0.116 mol) in 200 ml of absolute dioxane LiAlH₄ (7.6 g; 0.2 mol) was added and the mixture boiled under argon for 18 hours. Then a mixture of water (7.5 ml) and dioxane (40 ml) was added drop-wise to the reaction mixture. The suspension was mixed for 20 minutes and filtered trough a dense glass filter. The filtrate was evaporated and the residue was distilled on Büchi oven for distillation at 120° C. and 0.1 mbar. Yield 17.6 g (70%).

### ¹H NMR (300 MHz, CDCl₃): 1.7-1.9 (m, 4H); 2.18 (s, 3H); 2.25 (d, 2H); 2.48 (dd, 2H); 2.95 (m, 2H); 3.39 (s, 2H); 7.1-7.3 (m, 5H).

8-Methyl-3,8-diazabicyclo[3.2.1]octane (Intermediate compound)

To a degassed by argon solution of 3-benzyl-8-methyl-3,8-diazabicyclo[3.2.1]octane (17.6 g; 0.08 mol) in methanol (50 ml), 10% Pd/C (1.0 g) was added and hydrogen passed into reaction mixture for 24 hours. The catalyst was filtered off, the filtrate evaporated and the residue distilled on Büchi oven for distillation at 100° C. and 0.1 mbar. Yield 8.5 g (85%).

### ¹H NMR (300 MHz, CDCl₃): 1.6 (m, 2H); 1.86 (s, 1H); 1.9-2,0 (m, 2H); 2,17 (s, 3H); 2.53 (m, 2H); 2.9-3.0 (m, 4H). ¹³C NMR (75 MHz, CDCl₃): 24.73; 41.72; 52.10; 62.08.

Reference related to preparation of intermediate compounds 1-5: II Farmaco 55 (8), August 2000, Pages 553-562.

### Method A

3,4-Dichlorophenyl chloroformate (Intermediate)

A mixture of phosgen (22.8 g, 46.0 mmol, 20% in toluene) and dichloromethane (25 ml) was cooled to 0° C. A mixture of 3,4-dichlorophenol (1.5 g, 9.2 mmol) in dichloromethane (25 ml) and pyridine (1.3 ml, 16.6 mmol) was added dropwise at the same temperature. The mixture was allowed to slowly reach room-temperature and was stirred for 15 h. The mixture was evaporated, co-evaporated with toluene. The crude product was used in method B without further purification.

### Method B

9-Methyl-3,9-diaza-bicyclo[3.3.1]nonane-3-carboxylic acid 3,4-dichloro-phenyl ester free base

9-Methyl-3,9-diazabicyclo[3.3.1]nonane (0.83 g, 5.91 mmol) was added to a mixture of 3,4-dichlorophenyl chloroformate (2.0 g, 8.87 mmol) and DME (40 ml). The mixture was stirred at room-temperature for 15 h. Aqueous ammonia (5 ml, conc) and water (50 ml) was added followed by extraction with dichloromethane (2×50 ml), dried and evaporated. Chromatography on silica gel with dichloromethane, 2.25% methanol and 0.25% aqueous ammonia as solvent gave the compound as free base. Yield 1.26 g (64%).

LC-ESI-HRMS of [M+H]+ shows 329,0833 Da. Calc. 329,082359 Da, dev. 2,9 ppm
9-Methyl-3,9-diaza-bicyclo[3.3.1]nonane-3-carboxylic acid 4-bromo-phenyl ester hydrochloric acid salt

Was prepared according to method A and B.

LC-ESI-HRMS of [M+H]+ shows 339.071 Da. Calc. 339.070816 Da, dev. 0.5 ppm
9-Methyl-3,9-diaza-bicyclo[3.3.1]nonane-3-carboxylic acid 6-methoxynaphthalen-2-yl ester free base

Was prepared according to method A and B.

LC-ESI-HRMS of [M+H]+ shows 341,1849 Da. Calc. 341,186518 Da, dev. −4,7 ppm
9-Methyl-3,9-diaza-bicyclo[3.3.1]nonane-3-carboxylic acid 2-oxo-2H-chromen-7-yl ester free base

Was prepared according to method A and B.

9-Methyl-3,9-diaza-bicyclo[3.3.1]nonane-3-carboxylic acid 2-oxo-2H-chromen-6-yl ester free base

Was prepared according to method A and B.

LC-ESI-HRMS of [M+H]+ shows 329.1508 Da. Calc. 329.150133 Da, dev. 2 ppm
8-Methyl-3,8-diaza-bicyclo[3.2.1]octane-3-carboxylic acid 4-bromo-phenyl ester hydrochloric acid salt

Was prepared according to method A and B from 8-methyl-3,8-diazabicyclo[3.2.1]-octane.

LC-ESI-HRMS of [M+H]+ shows 325.054 Da. Calc. 325.055166 Da, dev. −3.6 ppm
8-Methyl-3,8-diaza-bicyclo[3.2.1]octane-3-carboxylic acid 2-oxo-2H-chromen-7-yl ester free base

Was prepared according to method A and B from 8-methyl-3,8-diazabicyclo[3.2.1]-octane.

LC-ESI-HRMS of [M+H]+ shows 315.1355 Da. Calc. 315.134483 Da, dev. 3.2 ppm

8-Methyl-3,8-diaza-bicyclo[3.2.1]octane-3-carboxylic acid 2-oxo-2H-chromen-6-yl ester free base

Was prepared according to method A and B from 8-methyl-3,8-diazabicyclo[3.2.1]octane.

LC-ESI-HRMS of [M+H]+ shows 315.1347 Da. Calc. 315.134483 Da, dev. 0.7 ppm

### Method C

3,9-Diaza-bicyclo[3.3.1]nonane-3-carboxylic acid 3,4-dichloro-phenyl ester hydrochloric acid salt

9-Methyl-3,9-diaza-bicyclo[3.3.1]nonane-3-carboxylic acid 3,4-dichlorophenyl ester free base (0.45 g, 1.35 mmol) and toluene (60 ml) was heated in a Dean and Stark water separator. The mixture was allowed to cool to room-temperature. 2,2,2-Trichloroethylchloroformate (2.58 g, 12.2 mmol) was added and the resulting mixture was stirred at reflux for 15 h. The mixture was washed with water and evaporated. The resulting intermediate was combined with acetic acid (10 ml), water (0.8 ml) and zinc powder (0.177 g, 2.71 mmol) and was stirred for 15 h at room-temperature. Additional zinc powder (0.531 g, 8.13 mmol) was added followed by stirring for 15 h at room-temperature. The mixture was filtered and evaporated followed by addition of HCl in diethylether (2 ml, 1 M). Yield 100 mg (21%).

LC-ESI-HRMS of [M+H]+ shows 315.0665 Da. Calc. 315.066709 Da, dev. −0.7 ppm
3,9-Diaza-bicyclo[3.3.1]nonane-3-carboxylic acid 2-oxo-2H-chromen-7-yl ester free base

Was prepared according to method C from 8-methyl-3,8-diaza-bicyclo[3.2.1]octane-3-carboxylic acid 2-oxo-2H-chromen-7-yl ester free base.

LC-ESI-HRMS of [M+H]+ shows 315.1334 Da. Calc. 315.134483 Da, dev. −3.4 ppm
3,8-Diaza-bicyclo[3.2.1]octane-3-carboxylic acid 2-oxo-2H-chromen-7-yl ester free base

Was prepared according to method C from 8-methyl-3,8-diaza-bicyclo[3.2.1]octane-3-carboxylic acid 2-oxo-2H-chromen-7-yl ester free base.

LC-ESI-HRMS of [M+H]+ shows 301,119 Da. Calc. 301,118833 Da, dev. 0.6 ppm

### Method D

9-Methyl-3,9-diaza-bicyclo[3.3.1]nonane-3-carboxylic acid 3-bromo-2-oxo-2H-chromen-7-yl ester free base
9-Methyl-3,9-diaza-bicyclo[3.3.1]nonane-3-carboxylic acid 2-oxo-2H-chromen-7-yl ester free base (0.50 g, 1.52 mmol) was solved in acetic acid (15 ml), followed by drop-wise addition of bromine (0.27 g, 1.67 mmol). The mixture was stirred for 15 h. Aqueous sodium hydroxide (30 ml, 1 M) was added followed by extraction with dichloromethane (2×20 ml). The mixture was dried and evaporated. Chromatography on silica gel with dichloromethane, 2-5% methanol and 1% aqueous ammonia as solvent gave the compound as free base. Yield 70 mg (11%).
LC-ESI-HRMS of [M+H]+ shows 407.061 Da. Calc. 407.060646 Da, dev. 0.9 ppm
In vitro Inhibition Activity

Compounds were tested for their ability to inhibit the reuptake of the monoamine neurotransmitters dopamine (DA) noradrenaline (NA) and serotonine (5-HT) in synaptosomes as described in WO 97/16451 (NeuroSearch A/S).

The test values are given as IC₅₀ (the concentration (μM) of the test substance which inhibits the specific binding of ³H-DA, ³H-NA, or ³H-5-HT by 50%).

Test results obtained by testing compounds of the present invention appear from the below table:

TABLE 1

| | 5-HT-uptake | DA-uptake | NA-uptake |
|---|---|---|---|
| Test compound | IC₅₀(μM) | IC₅₀(μM) | IC₅₀(μM) |
| 9-Methyl-3,9-diaza-bicyclo[3.3.1]nonane-3- | 0.34 | 0.030 | 0.024 |
| carboxylic acid 6-methoxynaphthalen-2-yl ester | | | |
| 9-Methyl-3,9-diaza-bicyclo[3.3.1]nonane-3- | 0.04 | 0.61 | 1.6 |
| carboxylic acid 2-oxo-2H-chromen-7-yl ester | | | |
| 3,9-Diaza-bicyclo[3.3.1]nonane-3-carboxylic | 0.0078 | >1.0 | 2.0 |
| acid 3,4-dichloro-phenyl ester | | | |
| 3,8-Diaza-bicyclo[3.2.1]octane-3-carboxylic | 0.027 | 0.092 | 0.62 |
| acid 2-oxo-2H-chromen-7-yl ester | | | |

From the foregoing it will be appreciated that, although specific embodiments of the invention have been described herein for purposes of illustration, various modifications may be made without deviating from the spirit and scope of the invention. Accordingly, the invention is not to be limited as by the appended claims.

The features disclosed in the foregoing description, in the claims and/or in the accompanying drawings, may both separately and in any combination thereof, be material for realising the invention in diverse forms thereof.

1. A compound of Formula I: 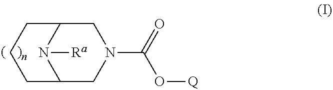
any of its stereoisomers or any mixture of its stereoisomers, or an N-oxide thereof, or a pharmaceutically acceptable salt thereof, wherein
n is 0 or 1;
R^{a} represents hydrogen or C₁₋₆-alkyl;
Q represents an aryl group or a chromen-2-one group, which aryl group or chromen-2-one group is optionally substituted with one or more substituents independently selected from the group consisting of halo, trifluoromethyl, trifluoromethoxy, cyano, hydroxy, amino, nitro, C₁₋₆-alkoxy, C₃₋₇-cycloalkoxy, C₁₋₆-alkyl, C₃₋₇-cycloalkyl, C₂₋₆-alkenyl and C₂₋₆-alkynyl;
and with the proviso that the compound is not 8-methyl-3,8-diaza-bicyclo[3.2.1]octane-3-carboxylic acid phenyl ester.

2. The compound according to claim 1, any of its stereoisomers or any mixture of its stereoisomers, or an N-oxide thereof, or a pharmaceutically acceptable salt thereof, wherein n is 0.

3. The compound according to claim 1, any of its stereoisomers or any mixture of its stereoisomers, or an N-oxide thereof, or a pharmaceutically acceptable salt thereof, wherein n is 1.

4. The compound according to claim 1, any of its stereoisomers or any mixture of its stereoisomers, or an N-oxide thereof, or a pharmaceutically acceptable salt thereof, wherein R^{a} represents hydrogen.

5. The compound according to claim 1, any of its stereoisomers or any mixture of its stereoisomers, or an N-oxide thereof, or a pharmaceutically acceptable salt thereof, wherein R^{a} represents C₁₋₆-alkyl.

6. The compound according to claim 1, any of its stereoisomers or any mixture of its stereoisomers, or an N-oxide thereof, or a pharmaceutically acceptable salt thereof, wherein Q represents an optionally substituted phenyl group.

7. The compound according to claim 1, any of its stereoisomers or any mixture of its stereoisomers, or an N-oxide thereof, or a pharmaceutically acceptable salt thereof, wherein Q represents an optionally substituted naphthyl group.

8. The compound according to claim 1, any of its stereoisomers or any mixture of its stereoisomers, or an N-oxide thereof, or a pharmaceutically acceptable salt thereof, wherein Q represents an optionally substituted chromen-2-one group.

9. The compound according to claim 1, which is
9-Methyl-3,9-diaza-bicyclo[3.3.1]nonane-3-carboxylic acid 3,4-dichloro-phenyl;
9-Methyl-3,9-diaza-bicyclo[3.3.1]nonane-3-carboxylic acid 4-bromo-phenyl ester;
9-Methyl-3,9-diaza-bicyclo[3.3.1]nonane-3-carboxylic acid 6-methoxynaphthalen-2-yl ester;
9-Methyl-3,9-diaza-bicyclo[3.3.1]nonane-3-carboxylic acid 2-oxo-2H-chromen-7-yl ester;
9-Methyl-3,9-diaza-bicyclo[3.3.1]nonane-3-carboxylic acid 2-oxo-2H-chromen-6-yl ester;
8-Methyl-3,8-diaza-bicyclo[3.2.1]octane-3-carboxylic acid 4-bromo-phenyl ester;
8-Methyl-3,8-diaza-bicyclo[3.2.1]octane-3-carboxylic acid 2-oxo-2H-chromen-7-yl ester;
8-Methyl-3,8-diaza-bicyclo[3.2.1]octane-3-carboxylic acid 2-oxo-2H-chromen-6-yl ester;
3,9-Diaza-bicyclo[3.3.1]nonane-3-carboxylic acid 3,4-dichloro-phenyl ester;
3,9-Diaza-bicyclo[3.3.1]nonane-3-carboxylic acid 2-oxo-2H-chromen-7-yl ester;
3,8-Diaza-bicyclo[3.2.1]octane-3-carboxylic acid 2-oxo-2H-chromen-7-yl ester;
9-Methyl-3,9-diaza-bicyclo[3.3.1]nonane-3-carboxylic acid 3-bromo-2-oxo-2H-chromen-7-yl ester;
any of its stereoisomers or any mixture of its stereoisomers, or an N-oxide thereof, or a pharmaceutically acceptable salt thereof.

10. A pharmaceutical composition, comprising a therapeutically effective amount of a compound according to claim 1, any of its stereoisomers or any mixture of its stereoisomers, or an N-oxide thereof, or a pharmaceutically acceptable salt thereof, together with at least one pharmaceutically acceptable carrier, excipient or diluent.

11. (canceled)

12. (canceled)

13. (canceled)

14. A method for the treatment, prevention or alleviation of a disease or a disorder or a condition of a living animal body, including a human, which disorder, disease or condition is responsive to inhibition of monoamine neurotransmitter re-uptake in the central nervous system, which method comprises the step of administering to such a living animal body in need thereof a therapeutically effective amount of a compound according to claim 1, or any of its stereoisomers or any mixture of its stereoisomers, or an N-oxide thereof, or a pharmaceutically acceptable salt thereof.

15. (canceled)

16. (canceled)

17. The method according to claim 14, wherein the disease, disorder or condition is mood disorder, depression, atypical depression, depression secondary to pain, major depressive disorder, dysthymic disorder, bipolar disorder, bipolar I disorder, bipolar II disorder, cyclothymic disorder, mood disorder due to a general medical condition, substance-induced mood disorder, pseudodementia, Ganser's syndrome, obsessive compulsive disorder, panic disorder, panic disorder without agoraphobia, panic disorder with agoraphobia, agoraphobia without history of panic disorder, panic attack, memory deficits, memory loss, attention deficit hyperactivity disorder (ADHD), obesity, anxiety, generalized anxiety disorder, eating disorder, Parkinson's disease, parkinsonism, dementia, dementia of ageing, senile dementia, Alzheimer's disease, Down's syndrome, acquired immunodeficiency syndrome dementia complex, memory dysfunction in ageing, specific phobia, social phobia, social anxiety disorder, post-traumatic stress disorder, acute stress disorder, drug addiction, drug abuse, drug abuse liability, cocaine abuse, nicotine abuse, tobacco abuse, alcohol addiction, alcoholism, kleptomania, withdrawal symptoms caused by termination of use of addictive substances, pain, chronic pain, inflammatory pain, neuropathic pain, migraine pain, tension-type headache, chronic tension-type headache, pain associated with depression, fibromyalgia, arthritis, osteoarthritis, rheumatoid arthritis, back pain, cancer pain, irritable bowel pain, irritable bowel syndrome, post-operative pain, post-mastectomy pain syndrome (PMPS), post-stroke pain, drug-induced neuropathy, diabetic neuropathy, sympathetically-maintained pain, trigeminal neuralgia, dental pain, myofacial pain, phantom-limb pain, bulimia, premenstrual syndrome, premenstrual dysphoric disorder, late luteal phase syndrome, post-traumatic syndrome, chronic fatigue syndrome, persistent vegetative state, urinary incontinence, stress incontinence, urge incontinence, nocturnal incontinence, sexual dysfunction, premature ejaculation, erectile difficulty, erectile dysfunction, premature female orgasm, restless leg syndrome, periodic limb movement disorder, eating disorders, anorexia nervosa, sleep disorders, pervasive developmental disorders, autism, Asperger's disorder, Rett's disorder, childhood disintegrative disorder, learning disabilities, motor skills disorders, mutism, trichotillomania, narcolepsy, post-stroke depression, stroke-induced brain damage, stroke-induced neuronal damage, Gilles de la Tourettes disease, tinnitus, tic disorders, body dysmorphic disorders, oppositional defiant disorder or post-stroke disabilities.
